(19) United States
(12) Patent Application Publication
(10) Pub. No.: US 2008/0248140 A1
(43) Pub. Date: Oct. 9, 2008

(54) COMPOSITION FOR SUPPORTING RESTFUL SLEEP

(76) Inventors: MARVIN HEUER, MISSISSAUGA (CA); KEN CLEMENT, MISSISSAUGA (CA); CHAUDHURI SHAN, MISSISSAUGA (CA); MEGAN THOMAS, MISSISSAUGA (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/696,948

(22) Filed: Apr. 5, 2007

(51) Int. Cl. A61K 36/84; A61P 43/00

(52) U.S. Cl. 424/733

(57) ABSTRACT

A method for promoting and supporting restful, quality sleep in an individual comprising the administration of a composition comprising Valerian root extract and Methylcobalamin.

### FIELD OF THE INVENTION

The present invention relates to supplemental compositions and methods for promoting and supporting restful sleep in an individual via the induction of sedation and melatonin support.

### BACKGROUND OF THE INVENTION

Sleep occupies about one-third of our life and is necessary for mental and physical well-being. It additionally affects mood, behavior and physiology. The need for sleep is a biological drive similar to thirst or hunger. Interestingly though, the function of sleep is largely unknown, however some evidence indicates that sleep is required for learning. It is clear that the impairment of sleep is detrimental to one's health. In humans, similarly, mild sleep deprivation also results in indications of impaired immune system function (Irwin M, McClintick J, Costlow C, Fortner M, White J, Gillin J C. Partial night sleep deprivation reduces natural killer and cellular immune responses in humans. FASEB J. 1996 April; 10(5):643-53). Prolonged sleep deprivation is even known to result in death. It has been determined my many that an individual can survive longer without food than one can without sleep; thus indicating the importance of sleep. Additionally, insomnia is estimated to affect a significant portion of North Americans per year and is associated with health problems and economic loss (Stoller M K. Economic effects of insomnia. Clin Ther. 1994 September-October; 16(5):873-97 Abstract).

Sleep as well as the control of sleep are complex processes involving multiple neuro-chemical pathways and associated brain structures. It is a shift in the balance of distinct physiological changes, involving both distinct physiological changes and involves both positive and negative neural signaling. The regulation of sleep in humans is governed by three processes—each influenced by hormonal and environmental factors: a daily sleep-wake cycle influenced by a circadian rhythm (24 hour cycle) tied to light-dark cycles.

Strategies to improve sleep are beneficial, not only in terms of physical health, but also in terms of emotional health. Furthermore, reinforcement of sleep of adequate quantity and quality positively impacts most aspect of daily life.

### SUMMARY OF THE INVENTION

The foregoing needs and other needs and objectives that will become apparent for the following description are achieved in the present invention, which comprises Valerian root extract and Methylcobalamin for the support of restful sleep in an individual via the induction of sedation and providing anxiolytic compounds and melatonin support.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a composition for supporting a restful period of sleep via the induction of sedation and providing anxiolytic compounds comprising Valerian root extract and Methylcobalamin. A method for same also provided comprising the step of the administration to an individual a composition comprising an extract of Valerian root supplying valerinic acid and Methlycobalamin.

Incorporated herein by reference are the specifications of co-pending applications (filed contemporaneously with the present application) entitled “Composition for a Feeling of Relaxation” and “Composition for a Feeling of Calmness”.

It is herein understood that improvements in sleep may be both of a quantitative nature e.g. increased length of sleep, decreased time of sleep onset, and of a qualitative nature e.g. deeper, more restful undisturbed. It is further understood that improvements in sleep may also be both direct and indirect. For example, sleep will be directly improved by the administration of a substance which is known to reduce time to sleep onset. Sleep may be indirectly improved, for example, by the administration of a substance which is known to result in feelings of relaxation and calmness.

### Valerian Root Extract

Valerian (Valeriana officinalis) is a perennial plant native to North America, Asia and Europe. The root has been traditionally used as treatment for anxiety and insomnia. A meta-analysis of available studies suggest that Valerian root extract may improve sleep absent of side-effects (Bent S, Padula A, Moore D, Patterson M, Mehling W. Valerian for sleep: a systematic review and meta-analysis. Am J. Med. 2006 December; 119(12):1005-12 Abstract) acting particularly to decrease the time to sleep onset and improved sleep quality in individuals.

Valerian has been shown to modulate Gamma Aminobutyric Acid (GABA) receptors (Yuan C S, Mehendale S, Xiao Y, Aung H H, Xie J T, Ang-Lee M K. The gamma-aminobutyric acidergic effects of valerian and valerenic acid on rat brainstem neuronal activity. Anesth Analg. 2004 February; 98(2):353-8). The neurotransmitter GABA, is the primary inhibitory neurotransmitter of the Central Nervous system. One of its effects is to induce sleep. Signaling through the GABA-receptor changes the electrochemical gradient of a given neuron, leading to inhibition of its activity. Benzodiazepines are thought to act via interaction with the GABA receptor; enhancing the inhibitory effects of GABA. As such, benzodiazepines are a widely used class of drugs primarily used as tranquilizers, muscle-relaxants, hypnotics or sedatives. It is believed that active constituents of Valerian produce sleep-enhancing sedative effects through this mechanism (Hadley S, Petry J J. Valerian. Am Fam Physician. 2003 Apr. 15; 67(8):1755-8).

Valerinic acid specifically has been shown to decrease neural activity in the brainstem produced by the GABA receptor antagonist, muscimol. Data from this study suggests that the pharmacological effects of valerian extract and valerenic acid are mediated through modulation of GABA(A) receptor function (Yuan C S, Mehendale S, Xiao Y, Aung H H, Xie J T, Ang-Lee M K. The gamma-aminobutyric acidergic effects of valerian and valerenic acid on rat brainstem neuronal activity. Anesth Analg. 2004 February; 98(2):353-8).

In a preferred embodiment of the present invention which is set forth in greater detail in the example below, the composition includes an extract of Valerian root having valerinic acid as an active component to promote a sedation leading to sleep through a GABAergic-dependant mechanism. A serving of the supplemental composition includes from about 0.001 g to about 1.000 g of an extract of Valerian root. The preferred dosage of an extract of Valerian root in the present invention comprises about 0.100 g per serving.

### Vitamin B12 (Methylcobalamin)

Vitamin B12 is a water-soluble cofactor for the enzyme methionine synthase, which transfers methyl groups. There are two forms of Vitamin B12-Adenosylcobalamin which is usually present in tissue and Methylcobalamin which is usually present in plasma. Vitamin B12 plays an important role in many important biological processes (Volkov I, Press Y, Rudoy I. Vitamin B12 could be a “master key” in the regulation of multiple pathological processes. J Nippon Med Sch. 2006 April; 73(2):65-9).

One specific area of vitamin B12 research pertains to the treatment of sleep disturbances. The Methylcobalamin form of vitamin B12 has been shown to be associated with increases in the quality of sleep and feelings of post-sleep restfulness (Mayer G, Kroger M, Meier-Ewert K. Effects of vitamin B12 on performance and circadian rhythm in normal subjects. Neuropsychopharmacology. 1996 November; 15(5):456-64). The mode of action of the benefit of vitamin B12 to sleep may be via support of melatonin synthesis (Methylcobalamin. Altern Med Rev. 1998 December; 3(6):461-3).

Methylcobalamin, as noted above, plays a role in melatonin production. Melatonin is the hormone responsible for the proper cycling of normal diurnal rhythms, or the circadian rhythm. As people get older, the body is less efficient at making this hormone. B12 supplementation has been shown to help some older adults sleep better, most likely through an increase in melatonin production.

In a preferred embodiment of the present invention which is set forth in greater detail in the example below, the composition includes Methylcobalamin to promote quality sleep by supporting the endogenous function of melatonin. A serving of the supplemental composition includes from about 0.000001 g to about 0.00001 g of Methylcobalamin. The preferred dosage of Methylcobalamin in the present invention comprises about 0.000006 g per serving.

In a preferred embodiment of the present invention, the composition comprises an extract of Valerian root and Methylcobalamin for the support of a restful period of sleep in an individual via the induction of sedation and melatonin support to maintain circadian rhythms relative to local time of a 24-hour day.

Not wishing to be bound by theory, it is believed that the components of the present invention will act in concert through distinct mechanisms to promote and support a restful period of sleep. The extract of Valerian root will act to promote sleep through the modulation GABA-receptors providing sedative and anxiolytic effect through GABA receptor agonism, while Methylcobalamin will act to support the normal endogenous activity of melatonin to regulate sleep according to a 24-hours system.

The composition of the present invention may be provided in various embodiments, however, the nutritional supplement resultant therefrom said embodiments may be administered to an individual in acceptable any form. For instance, the dosage form of the nutritional supplement may be provided as, e.g., a powder beverage mix, a liquid beverage, a ready-to-eat bar or drink product, a capsule, a liquid capsule, a tablet, a caplet, a soft-gel capsule or as a dietary gel. The preferred dosage forms of the present invention are as a tablet or caplet.

An embodiment of the present invention the composition may be provided in a solid dosage form having specific controlled release characteristics. Advantageously, the composition may be provided in a layered solid dosage form. In such a form, each individual layer will provide unique dissolution characteristics. In this way a controlled release of the composition can be achieved. In one aspect of this embodiment, each layer contains a homogeneous mixture of ingredients whereby the release of all ingredients is dependent upon the characteristics of each given layer. In an alternative aspect of this embodiment, each layer contains a distinct set of specific ingredients which differ according to the layers such that different specific ingredients are released from the solid dosage form at different times according to a predetermined schedule. In all aspects of this embodiment, a temporally controlled release of ingredients is achieved.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art may be utilized in methods to promote and support a restful period of sleep in an individual.

Advantageously, the present composition may be used not only as a sole means of promoting and supporting restful sleep but may also be used either in addition to other similarly-directed compositions or as a component of a larger composition. Furthermore, as a component of a greater composition the present invention, may serve to provide a given aspect contributing to the function of broader composition wherein other requirements for good sleep in an individual are addressed by additional compositions.

Additionally, by way of ingestion of the composition of the present invention, a method for improving the onset of sleep and improvement of sleep quality is provided. The method of the present invention comprises at least the step of administering to an individual a therapeutically acceptable amount of the composition of the present invention.

Although the following example illustrates the practice of the present invention in one of its example compositional embodiments, the example should not be construed as limiting the scope of the invention. Other embodiments will be apparent to one of skill in the art from consideration of the specifications and example.

### EXAMPLE

A nutritional supplement to help promote and support restful, quality sleep for use immediately prior to bedtime. A serving of the nutritional supplement as a caplet contains the following:

About 0.1000 g of an extract of Valerian root standardized to Valerinic acid and about 0.000006 g of Methycobalamin.

### Extensions and Alternatives

In the foregoing specification, the invention has been described with a specific embodiment thereof, however, it will be evident that various modifications and changes may be made thereto without departing from the broader spirit and scope of the invention.

What is claimed:

1. A composition comprising an effective amount of an extract of Valerian root and an effective amount Methylcobalamin for promoting sleep in a mammal.

2. The composition of claim 1 wherein the extract of Valerian root agonizes GABA-receptors to induce sedation in a mammal.

3. The composition of claim 1 wherein the extract of Valerian root is standardized to Valerinic Acid.

4. The composition of claim 1 wherein the Methylcobalamin supports the endogenous function of melatonin to maintain normal diurnal rhythms according to a 24-hour cycle.

5. A method of promoting sleep in an individual, the method comprising the step of administering to a mammal a therapeutically effect amount of a composition comprising an extract of Valerian root and Methylcobalamin.

6. The method of claim 5 wherein the extract of Valerian root agonizes GABA-receptors to induce sedation in a mammal.

7. The method of claim 6 wherein the extract of Valerian root is standardized to Valerinic Acid.

8. The method of claim 5 wherein the Methylcobalamin supports the endogenous function of melatonin to maintain normal diurnal rhythms according to a 24-hour cycle.

9. The composition of claim 1, wherein the composition is provided in a layered release solid dosage form.

10. The method of claim 5, wherein the composition is provided in a layered release solid dosage form.
